(19) United States
(12) Patent Application Publication
(10) Pub. No.: US 2024/0226557 A9
(43) Pub. Date: Jul. 11, 2024

(54) HIGH-BANDWIDTH SYSTEMS FOR CLOSED-LOOP SIGNAL CORD STIMULATION

(71) Applicant: PRECISION NEUROSCIENCE CORPORATION, New York, NY (US)

(72) Inventors: Benjamin I. RAPOPORT, New York, NY (US); Craig H. MERMEL, New York, NY (US); Daniel TRIETSCH, New York, NY (US); Mark HETTICK, New York, NY (US); Elton HO, New York, NY (US); Kazutaka TAKAHASHI, New York, NY (US)

(73) Assignee: PRECISION NEUROSCIENCE CORPORATION, New York, NY (US)

(21) Appl. No.: 18/491,333

(22) Filed: Oct. 20, 2023

Related U.S. Application Data
(60) Provisional application No. 63/417,922, filed on Oct. 20, 2022.

(51) Int. Cl. A61N 1/36; A61N 1/02

(52) U.S. Cl. CPC A61N 1/36062; A61N 1/025; A61N 1/36067; A61N 1/36071; A61N 1/36135; A61N 1/0553

(57) ABSTRACT

A system for performing spinal cord stimulation on a subject is disclosed. The system comprises one or more recording arrays, at least one stimulation array, and a processor. The recording arrays, which may be minimally invasively inserted to a target recording site of the brain, include recording electrodes having a diameter of less than about 1 mm and having a spacing of less than about 1 mm therebetween. The stimulation array, which may be minimally invasively inserted to a target stimulation site within the nervous tissue, includes stimulation electrodes. The processor is configured to receive more recorded signals from the recording arrays at the target recording site, determine an electrophysiological state of the brain based on the recorded signals, and deliver electrical stimulation to the target stimulation site through the stimulation array based on the determined electrophysiological state in order to affect the electrophysiological state.

### PRIORITY

The present application claims priority to U.S. Provisional Patent Application No. 63/417,922, titled HIGH-BANDWIDTH SYSTEMS FOR CLOSED-LOOP SPINAL CORD STIMULATION, filed Oct. 20, 2022, which is hereby incorporated by reference herein in its entirety.

### TECHNICAL FIELD

The present disclosure relates generally to methods, systems, and apparatuses related to performing spinal cord stimulation on a subject. More particularly, the present disclosure relates to a high-bandwidth system for performing closed-loop spinal cord stimulation. The disclosed techniques may be applied to, for example, treating neurological disorders and/or injuries associated with the spinal cord, such as multiple system atrophy, paralysis, autonomic dysfunction, and chronic pain.

### BACKGROUND

Spinal cord stimulation (SCS) is a technique used for treating certain forms of chronic pain (e.g., as an alternative to long-term narcotic medications) and increasingly for other disorders or injuries associated with the spinal cord, such as multiple system atrophy, paralysis, and autonomic dysfunction. SCS involves epidural electrical stimulation of functional targets within the spinal cord or spinal nerve roots to alter their electrophysiologic activity in a controlled fashion.

Traditional implementations of the technique operate in an “open-loop” fashion in which a target tissue is stimulated continuously, and therapeutic parameters such as frequency and amplitude of stimulation do not vary on the basis of disorder state, symptoms, side effects, or environmental factors. While known to be suboptimal, open-loop stimulation is a simplified approach that must be intermittently reprogrammed by an external user to change stimulation parameters.

The concept of “closed-loop” neuromodulation describes systems capable of both sensing of signals from a target tissue and stimulation in response thereto, i.e., the stimulation is adaptive and responds to sensed events or circumstances. Closed-loop systems hold the promise of providing more personalized and context-aware therapy with greater overall efficacy and fewer side effects. Closed-loop systems may also allow for improved device lifetimes because stimulation is targeted rather than continuous, thus utilizing implanted batteries in a more efficient manner. Closed-loop neuromodulation has seen some clinical adoption for targeting the deep brain to manage epilepsy by detecting the onset of a seizure through recording electrodes and selectively delivering electrical stimulation via stimulation electrodes in an adaptive manner to prevent the seizure from occurring. Clinical evidence suggests that closed-loop neuromodulation may also be effective for treating Parkinson's disease and/or treatment-resistant depression.

However, currently available closed-loop systems are not currently used for SCS applications; rather, all available solutions deliver stimulation in an open-loop fashion as described above on the basis of subjective symptoms and side effects reported by a patient. Furthermore, currently available closed-loop systems provide only coarse estimates of the electrophysiologic state of a subject. Indeed, neurons in mammals may be as small as tens of microns in diameter and clinically relevant brain states may be represented by the coordinated activity of hundreds or thousands of neurons. As such, electrophysiological activity may span spatial scales from fractions of a millimeter to a few millimeters. On the other hand, conventional systems typically utilize electrode arrays comprising a small number of large electrodes that provide low spatial resolution (see, for example, FIG. 4), which limits the systems' ability to accurately detect many electrophysiologic states and/or differentiate between similarly presenting electrophysiologic states.

Furthermore, closed-loop systems have not been thought to be practical for SCS because it would require implantation of electrodes are various locations of the nervous system (e.g., the spinal cord, one or more regions of the brain, etc.), which requires highly invasive and potentially damaging surgical procedures. Ultimately, the degree of invasiveness and collateral damage is a limiting factor on the number of recording electrodes that may feasibly be applied to a subject and, consequently, the amount of data that may be collected for assessment of electrophysiological state. Still further, the magnitude of the procedures results in a limited ability to adjust the spatial placement of the electrodes after initial placement.

Accordingly, it would be advantageous to have a system for providing closed-loop SCS using high-bandwidth neural electrode arrays in order capture the dynamic electrophysiologic state of the nervous system from one or more locations with high spatial resolution and provide stimulation to the nervous tissue in an adaptive fashion.

### SUMMARY

A system for performing spinal cord stimulation on a patient is provided. The system comprises: one or more recording arrays configured to be minimally invasively inserted to a target recording site of a brain of the patient, each recording array comprising a plurality of recording electrodes having a spacing of less than about 1 mm therebetween, each recording electrode having a diameter of less than about 1 mm and configured to record electrical signals from the target recording site; at least one stimulation array configured to be inserted to a target stimulation site proximate to nervous tissue of the patient, each stimulation array comprising a plurality of stimulation electrodes, each stimulation electrode configured to deliver electrical stimulation to the target stimulation site; one or more inertial sensors configured to detect at least one of an orientation and a movement state of the patient; a processor; and a non-transitory, computer-readable medium storing instructions that, when executed, cause the processor to: receive, via the one or more recording arrays, one or more recorded signals from the target recording site, determine an electrophysiological state of the brain based on at least the one or more recorded signals, receive one or more inertial signals from the one or more inertial sensors, determine the at least one of an orientation and a movement state of the patient based on the one or more inertial signals, and selectively deliver, via the at least one stimulation array, electrical stimulation to the target stimulation site based on one or more of the determined electrophysiological state and the determined at least one of an orientation and a movement state of the patient, wherein the set of electrical stimulation is configured to electrically stimulate the nervous tissue.

According to some embodiments, each of the one or more recording arrays is a thin-film electrode array.

According to some embodiments, each of the one or more recording arrays and the at least one stimulating array is a two-dimensional electrode array.

According to some embodiments, each of the one or more recording arrays has a spatial density of at least about 100 electrodes/cm².

According to some embodiments, each of the one or more recording arrays comprises at least about 100 electrodes. According to additional embodiments, each of the one or more recording arrays includes about 529 electrodes. According to additional embodiments, each of the one or more recording arrays includes about 1,024 electrodes.

According to some embodiments, the electrophysiological state is selected from chronic pain, autonomic dysfunction, autonomic dysfunction with dominant components of cardiac arrhythmia or intermittent hypotension, postural instability, gait dysfunction, multiple system atrophy, shaking, tremors, paralysis, blood pressure control, heart rate control, and postural control.

According to some embodiments, the electrophysiological state is tremors, shaking, or paralysis, and the target stimulation site is the spinal cord.

According to some embodiments, the electrophysiological state is pain, and the target stimulation site is a nervous tissue detecting pain, a nervous system tissue conducting pain signals to the brain, a nervous system tissue processing pain signals, or a combination thereof. According to additional embodiments, the electrophysiological state is pain at a level below a threshold of conscious awareness for the patient.

A computer-implemented method for performing spinal cord stimulation on a patient is also provided. The method comprises: receiving one or more recorded signals from a target recording site via one or more recording arrays positioned at the target recording site, each recording array comprising a plurality of recording electrodes having a spacing of less than about 1 mm therebetween, each recording electrode having a diameter of less than about 1 mm; receiving one or more inertial signals indicative of at least one of an orientation and a movement state of the patient via one or more inertial sensors; determining an electrophysiological state of the brain based on at least the one or more recorded signals; determining the at least one of an orientation and a movement state of the patient based on the one or more inertial signals; and selectively delivering, based on one or more of the determined electrophysiological state and the determined at least one of an orientation and a movement state, electrical stimulation to a target stimulation site via at least one stimulation array positioned at the target stimulation site to electrically stimulate the target stimulation site, wherein the target stimulation site is located at one of a spinal cord and spinal nerve roots of the patient.

According to some embodiments, each of the one or more recording arrays is a thin-film electrode array.

According to some embodiments, the one or more recording arrays include a subdural electrode array.

According to some embodiments, the one or more recording arrays include an epidural electrode array.

According to some embodiments, each of the one or more recording arrays and the at least one stimulating array is a two-dimensional electrode array.

According to some embodiments, each of the one or more recording arrays has a spatial density of at least about 100 electrodes/cm².

According to some embodiments, each of the one or more recording arrays comprises at least about 100 electrodes. According to additional embodiments, each of the one or more recording arrays includes about 1,024 electrodes.

According to some embodiments, the electrophysiological state is selected from chronic pain, autonomic dysfunction, autonomic dysfunction with dominant components of cardiac arrhythmia or intermittent hypotension, postural instability, gait dysfunction, multiple system atrophy, shaking, tremors, paralysis, blood pressure control, heart rate control, and postural control.

According to some embodiments, the electrophysiological state is tremors, shaking, or paralysis and the target stimulation site is the spinal cord.

According to some embodiments, the electrophysiological state is pain, and the target stimulation site is a nervous tissue detecting pain. According to additional embodiments, the electrophysiological state is pain at a level below a threshold of conscious awareness for the patient.

### BRIEF DESCRIPTION OF THE DRAWINGS

The accompanying drawings, which are incorporated in and form a part of the specification, illustrate the embodiments of the invention and, together with the written description, serve to explain the principles, characteristics, and features of the invention. Various aspects of at least one example are discussed below with reference to the accompanying drawings, which are not intended to be drawn to scale. In the drawings:

FIG. 1 depicts a diagram of an illustrative neural interface system communicatively coupled to an external device in accordance with an embodiment.

FIG. 2 depicts a subdural implanted neural device in accordance with an embodiment.

FIG. 3 depicts a detailed view of an electrode array of a neural device in accordance with an embodiment.

FIG. 4 depicts a conventional neuromodulation system 400 for spinal cord stimulation in accordance with an embodiment.

FIG. 5 depicts a block diagram of an illustrative system for performing closed-loop spinal cord stimulation in accordance with an embodiment.

FIG. 6A depicts a comparison of the stimulation array 510 of FIG. 5 to a conventional stimulation electrode array in accordance with an embodiment.

FIG. 6B depicts a comparison between a 529-channel recording array and a 1,024-channel recording array in accordance with an embodiment.

FIG. 7 depicts a flow diagram of an illustrative computer-implemented method for performing spinal cord stimulation in accordance with an embodiment.

FIG. 8 illustrates a block diagram of an exemplary data processing system in which embodiments are implemented.

### DETAILED DESCRIPTION

In this disclosure, improved spinal cord stimulation systems are described that are capable of delivering improved performance over existing systems and conventional recording electrodes. Currently, all spinal cord stimulation is performed in an “open-loop” manner that lacks dynamic changes in stimulation parameters with only intermittent reprogramming. There is, for example, no way for a spinal cord stimulation system to change parameters when a patient feels worse pain or when the brain begins registering pain that is yet below a threshold of conscious awareness. In this disclosure, closed-loop spinal cord stimulation systems are described with expected improvements in performance over conventional systems due to higher bandwidth electrophysiologic data that is able to be collected, the ability of the system to practicably interface with diverse and non-adjacent regions of the nervous system, the minimally invasive and reversible nature electrode placement, and the data-handling and computational capacity of the closed-loop neuromodulation system.

### Minimally Invasive Neural Interface Systems

The minimally invasive electrode arrays utilized by the systems herein are first described in greater detail. Neural interface systems can sense and record brain activity, receive instructions for stimulating the subject's brain, and otherwise interact with a subject's brain as generally described herein. In contrast to conventional systems that require significant surgery for insertion and may require penetration of the cortical surface, the neural interface systems described herein may utilize minimally invasive electrode arrays adapted for insertion with less damage to the body than open surgery and less impact on the subject's cortical tissue.

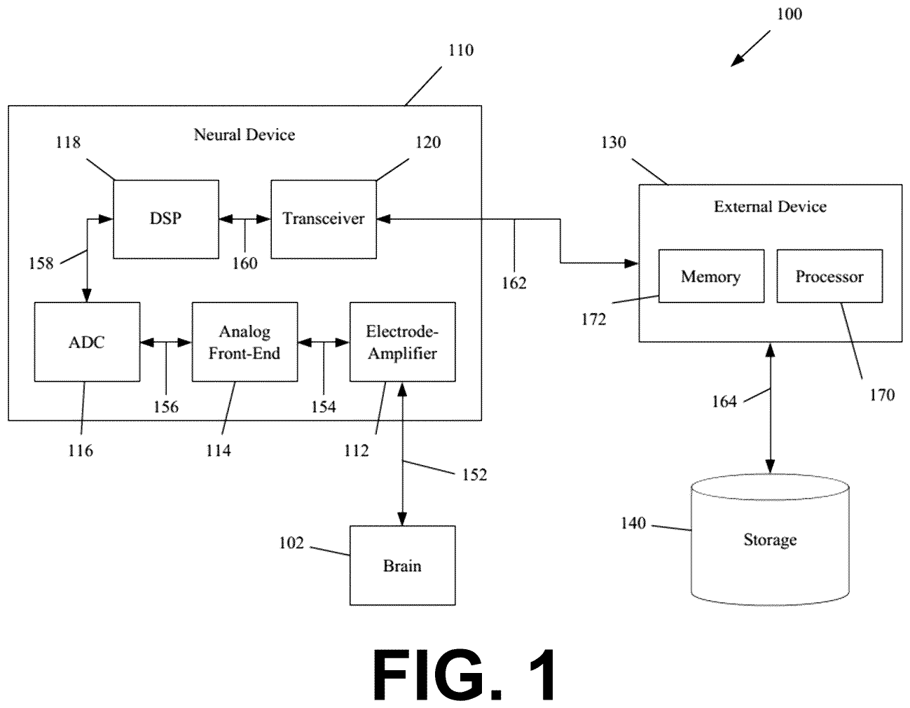

Referring now to FIG. 1, a diagram of an illustrative neural interface system 100 is depicted in accordance with an embodiment. The system 100 includes a neural device 110 that is communicatively coupled to an external device 130. The external device 130 may include any device with which the neural device 110 may be communicatively coupled such as a computer or mobile device, e.g., a tablet, a smartphone, a laptop, a desktop, a secure server, a smartwatch, a head-mounted virtual reality device, a head-mounted augmented reality device, or a smart inductive charger device. The external device 130 may include a processor 170 and a memory 172. In some embodiments, the external device 130 may include a server or a cloud-based computing system. In some embodiments, the external device 130 further comprises and/or is communicatively coupled to a storage unit 140. In one embodiment, the storage unit 140 comprises a database stored on the external device 130. In another embodiment, the storage unit 140 comprises a cloud computing system (e.g., Amazon Web Services or Azure).

The neural device 110 may include a range of electrical or electronic components. As shown in FIG. 1, in some embodiments, the neural device 110 includes an electrode-amplifier stage 112, an analog front-end stage 114, an analog-to-digital converter (ADC) stage 116, a digital signal processing (DSP) stage 118, and a transceiver stage 120 that are communicatively coupled to one another. The electrode-amplifier stage 112 may include an electrode array, as further described herein, that is capable of physically interfacing with the brain 102 of a subject in order to sense brain signals and/or apply electrical signals thereto. The analog front-end stage 114 may be configured to amplify signals that are sensed from or applied to the brain 102, perform conditioning of the sensed or applied analog signals, perform analog filtering, and so on. The front-end stage 114 may include, for example, one or more application-specific integrated circuits (ASICs) or other electronics. The ADC stage 116 may be configured to convert received analog signals to digital signals for processing via the analog front-end stage 114 and application via the electrode-amplifier stage 112. The DSP stage 118 can be configured to perform various DSP techniques, including multiplexing of digital signals received via the electrode-amplifier stage 112 and/or from the external device 130. For example, the DSP stage 118 may be configured to convert instructions from the external device 130 to a corresponding digital signal. The transceiver stage 120 may be configured to transfer data from the neural device 110 to the external device 130 located outside of the body of the subject.

In some embodiments, the neural device 110 can further include a controller 119 configured to perform various functions, including compressing electrophysiologic data generated by the electrode array 180. In various embodiments, the controller 119 can include hardware, software, firmware, or various combinations thereof that are operable to execute the functions described below. In one embodiment, the controller 119 can include a processor (e.g., a microprocessor) executing instructions stored in a memory. In another embodiment, the controller 119 can include a field-programmable gate array (FPGA) or application-specific integrated circuit (ASIC).

The electrode array 180 can include a series of electrodes that are arranged in an array or regularly spaced configuration. Accordingly, the electrode array 180 of the neural device 110 can be of a sufficient size to measure one or more areas of interest along the cortical surface. In one embodiment, the neural device 110 can include a number of electrodes (i.e., channels) that is sufficient to measure one or more areas of the cortical surface of interest. In various embodiments, the electrode array 180 could include about 100 or more electrodes, about 500 or more electrodes, or about 1,000 or more electrodes. For example, the electrode array 180 could include one thousand or more electrodes. In one illustrative embodiment, the electrode array 180 could include 1,024 electrodes.

In various embodiments, the stages of the neural device 110 could provide unidirectional or bidirectional communications. As indicated in FIG. 1, the external device 130 and the stages 112, 114, 116, 118, 120 of the neural device 110 may be electrically coupled by connectors 154, 156, 158, 160, 162, which may be electrical wires, busses, or any type of electrical connector that enables unidirectional or bidirectional communications as would be known to a person having an ordinary level of skill in the art. Furthermore, the electrode-amplifier stage 112 and the brain 102 of the subject may be electrically coupled by a connector 152, e.g., an electrode array as further described herein. In some embodiments, the connector 152 may be considered part of the electrode-amplifier stage 112. It can be understood that the depicted architecture for the system 100 is merely illustrative and the system 100 could be arranged in various different manners, i.e., stages or other components of the system 100 may be connected differently and/or the system 100 may include additional or alternate stages or components. For example, any of the stages may be arranged and operate in a serial or parallel fashion with other stages of the system 100.

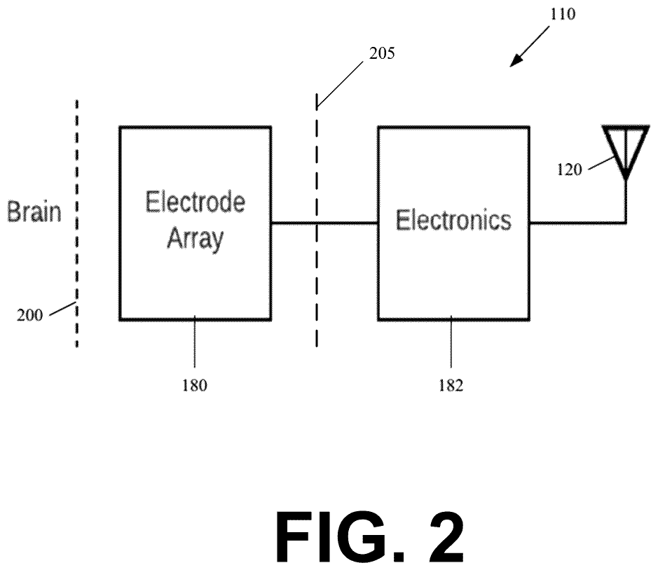

In some embodiments, the neural device 110 may be a neural implant, e.g., a biomedical device configured to study, investigate, diagnose, treat, and/or augment brain activity. As shown in FIG. 2, the neural device may be a subdural implanted neural device, i.e., a neural device implanted between the dura (i.e., the membrane surrounding the brain) and the cortical surface of the brain. In some embodiments, the neural device 110 may be positioned beneath the dura mater or between the dura mater and the arachnoid membrane. In some embodiments, the neural device 110 may be positioned in the subdural space, on the cortical surface of the brain 200. The neural device 110 may be inserted through an incision in the scalp 202 and across the dura. The neural device 110 may include an electrode array 180, which may be the connector 152 and/or a component of the electrode-amplifier stage 112 of FIG. 1. The electrode array 180 may be configured to record and/or stimulate a target site of the brain 200. The electrode array 180 may be connected to an electronics hub 182, which may include one or more components of the neural device 100, i.e., the electrode-amplifier stage 112, the analog front-end stage 114, the ADC stage 116, and/or the DSP stage 118. The electronics hub 182 may be configured to receive or transmit signals via wireless or wired transceiver 120 to the external device 130 (which may be referred to as a “receiver” herein).

The electrode array 180 may include cortical surface microelectrodes that are configured not to penetrate surface of the brain 200. Accordingly, the electrode array 180 is capable of interfacing with the brain 200 in a minimally invasive manner, thus expanding the applicability of the neural device 110 and enabling brain-computer interface technology for a larger population of patients than conventional neural implants. Furthermore, the surgical procedures for implanting the neural devices 110 may be minimally invasive, may be reversible, and may avoid damaging neural tissue. In some embodiments, the electrode array 180 may be a high-density microelectrode array that provides smaller features and improved spatial resolution relative to conventional neural implants.

In some embodiments, the neural device 110 further comprises one or more of control logic for operating the stages 112, 114, 116, 118, 120 of the neural device 110 and/or the electrode array 180, a memory for storing recordings from the electrode array and/or instructions for the electrode array, and a power management unit for providing power to various components of the neural device 110 such as the electrode array 180.

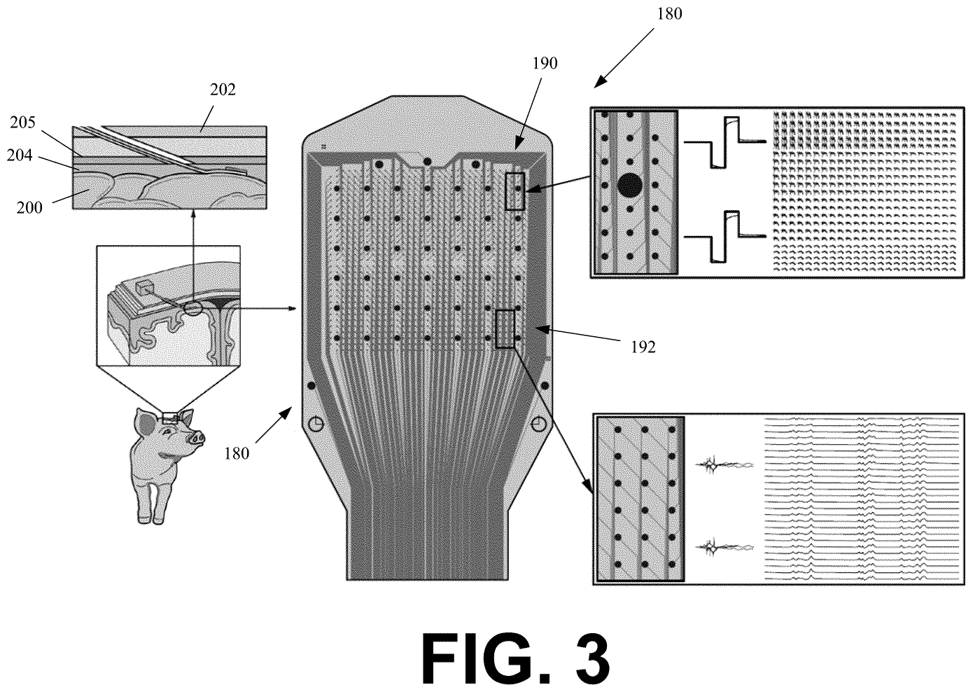

Referring now to FIG. 3, a detailed view of an electrode array 180 of a neural device 110 is depicted in accordance with an embodiment. As shown, the neural device 110 may include an electrode array 180 comprising non-penetrating microelectrodes. The microelectrodes of the electrode array 180 may be arranged in a variety of different configurations and may vary in size. In some embodiments, the electrodes of the electrode array 180 could be from about 10 μm to about 500 μm in width. In one illustrative embodiment, the electrodes of the electrode array 180 could be about 50 μm in width. In some embodiments, the electrodes of the electrode array 180 could be spaced by about 200 μm (i.e., 0.2 mm) to about 3,000 μm (i.e., 3 mm). In illustrative one embodiment, adjacent electrodes of the electrode array 180 could be spaced by about 400 μm. In various embodiments the electrode array 180 could include electrodes of the same or different sizes. For example, in the embodiment shown in FIG. 3, the electrode array 180 may include a first set 190 of electrodes (e.g., about 200 μm electrodes) and a second set 192 of electrodes (e.g., about 20 μm electrodes). In some embodiments, the electrode array 180 comprises a 1024-channel electrode array.

The electrode array 180 may be configured for minimally invasive subdural implantation, i.e., insertion into the subdural space 204 between the dura and the surface of the subject's brain 200. As shown in FIG. 3, the electrode array 180 may be delivered using a cranial micro-slit technique. For example, a surgeon may cut an angled slit (e.g., a micro-slit having a width of about 400 microns or less) through the cranium to provide access to the subdural space. Thereafter, the electrode array 180 may be inserted through the micro-slit and into the subdural space 204 in contact with the brain 200. Accordingly, the neural device 110 may provide high spatial-resolution with minimal invasiveness and improved signal quality.

In some embodiments, the neural device 110 may be fully implanted in the body. For example, the electrode array 180 may be connected to a self-contained, fully implantable unit that contains the remainder of the components of the neural device 110. In some embodiments, the neural device may include custom hardware and/or an application-specific integrated circuit (ASIC). The unit may have extensive data processing capability, as in the cases of pacemakers and deep brain stimulators, to enable closed-loop recording and stimulation. In another embodiment, the electrode array 180 may be connected by wires to a unit implanted elsewhere in the body (e.g., the chest wall). In another embodiment, the unit may be externally wearable or mounted on the body of the subject (e.g., on the scalp). In some embodiments, the unit may be a power-and-data unit that is connected to both the recording array and the stimulation array of the electrode array 180. In another embodiment, the unit may comprise two power-and-data units coupled together, wherein one of the power-and-data units is connected to the recording array of the electrode array 180, and the other power-and-data unit is connected to the stimulation array of the electrode array 180. In some embodiments, all of the electronics may be contained on the electrode array itself, with no wired connection to a secondary unit.

Additional information regarding brain-computer interfaces described herein can be found in Ho et al., The Layer 7 Cortical Interface: A Scalable and Minimally Invasive Brain—Computer Interface Platform, bioRxiv 2022.01.02.474656, doi:
https://doi.org/10.1101/2022.01.02.474656, which is hereby incorporated by reference in its entirety.

### High-Bandwidth Systems for Closed-Loop Spinal Stimulation

As discussed herein, closed-loop neuromodulation provides therapeutic electrical stimulation to the nervous system in response to estimates of a state of the nervous system on the basis of brain electrical activity. Closed-loop neuromodulation entails both sensing neural signals from the brain and providing stimulation to nervous tissue in an adaptive fashion based on sensed events or circumstances, thereby enabling more personalized and context-aware therapy with greater overall efficacy, fewer side effects, and improved device lifetimes.

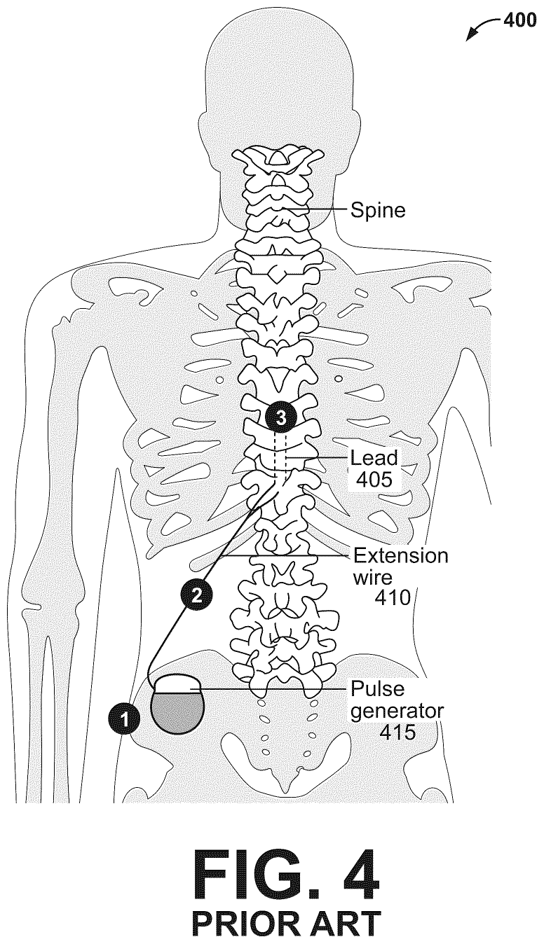

Clinically relevant brain states are represented by the activity of neurons spanning spatial scales from fractions of a millimeter to a few millimeters. However, current neuromodulation systems are limited to low-bandwidth recording arrays comprising small numbers of large electrodes with low spatial resolution. For example, FIG. 4 depicts a conventional neuromodulation system 400 in accordance with an embodiment. The system 400 may comprise an epidural stimulation lead 405 comprising stimulation electrodes at a stimulation site at the spine, an extension wire 410 communicating with the epidural stimulation lead 405, and a pulse generator unit 415 communicating with the extension wire 410 and configured to electrically communicate with the epidural stimulation lead 405 via the extension wire 410. Notably, the epidural stimulation lead 405 may comprise a small number of large electrodes (e.g., sixteen electrodes having a diameter greater than 1 mm) that are spaced by several millimeters, resulting in low resolution stimulation of the nervous tissue. The number of electrodes that may be placed is limited by the invasive procedures generally associated with electrode implantation. Furthermore, there are no recording electrodes to collect information related to the electrophysiological state of the subject. As such, it would be advantageous to have a system for providing closed-loop spinal cord stimulation (SCS) using high-bandwidth neural electrode arrays in order capture the dynamic electrophysiologic state of the brain surface in high spatial and temporal resolution and provide stimulation to the brain in an adaptive fashion.

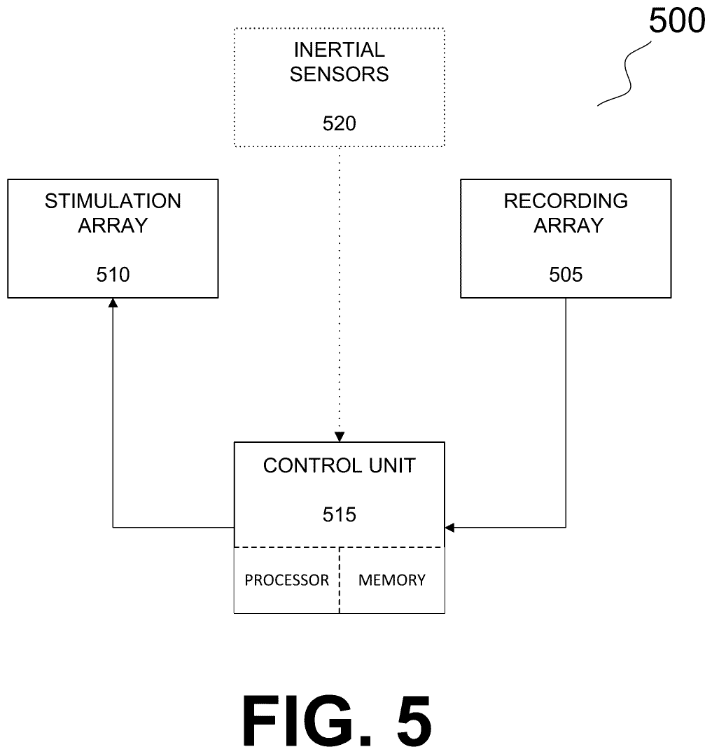

Turning now to FIG. 5, a block diagram of an illustrative system for performing closed-loop spinal cord stimulation is depicted in accordance with an embodiment. The system 500 comprises one or more recording arrays 505, at least one stimulation array 510, and a control unit 515 comprising a processor and a memory. The one or more recording arrays 505 and the at least one stimulation array 510 are each in electrical communication with the control unit 515. In some embodiments, the system 500 may further comprise one or more inertial sensors 520 in electrical communication with the control unit 515.

Each of the one or more recording arrays 505 may comprise a plurality of recording electrodes capable of recording electrical signals from a target recording site. The recording array 505 may be configured to be minimally invasively inserted to the target recording site of the brain as described herein. For example, the recording array 505 may be delivered to the subdural space and/or additional locations using a cranial micro-slit technique as described herein with respect to the electrode array 180. It can be understood that each recording array 505 may be a thin-film, two-dimensional electrode array such as the electrode array 180 as described herein with respect to FIGS. 2-3 and may comprise any of the features and/or functions as described with respect to the electrode array 180.

In some embodiments, each recording array 505 may be a high-density array that enables greater spatial resolution. For example, the recording electrodes may be arranged with a spatial density of about 100 electrodes/cm², about 200 electrodes/cm², about 300 electrodes/cm², about 400 electrodes/cm², about 500 electrodes/cm², or individual values or ranges therebetween. Accordingly, the recording electrodes may be spaced from one another by about 1 mm, about 500 μm, about 250 μm, about 100 μm, about 50 μm, less than about 50 μm, or individual values or ranges therebetween.

In some embodiments, the recording array 505 may comprise about 529 electrodes. In other embodiments, the recording array 505 may comprise about 1024 electrodes. However, it is contemplated that the recording array 505 may comprise about 100 electrodes, about 200 electrodes, about 300 electrodes, about 400 electrodes, about 500 electrodes, about 1000 electrodes, greater than 1000 electrodes, or individual values or ranges therebetween.

In some embodiments, the recording electrodes of each recording array 505 may be cortical surface microelectrodes that are configured not to penetrate the surface of the brain. In some embodiments, each recording electrode may have a diameter of less than about 1 mm. For example, each recording electrodes may have a diameter of about 50 μm. However, it is contemplated that the recording electrodes may have a diameter of about 20 μm, about 40 μm, about 60 μm, about 80 μm, about 100 μm, about 200 μm, about 300 μm, about 400 μm, about 500 μm, greater than about 500 μm, or individual values or ranges therebetween. In some embodiments, the recording electrodes may comprise combinations of the electrode sizes recited herein.

The recording array 505 as described herein may enable collection of higher bandwidth electrophysiologic data. Given that neuron cell bodies may be tens of microns in diameter, the size and arrangement of the recording electrodes enables the recording array 505 to sense signals from the target recording site with a spatial resolution at a similar scale to the neuron cell bodies. Furthermore, the recording array 505 may have a total number of electrodes that is several orders of magnitude greater than conventional recording arrays.

As described, in conventional systems, the invasive nature and the damage to the tissue associated with implantation generally restricts the number of recording arrays and/or the number of locations at which the recording arrays may be positioned. In contrast, the recording arrays 505 may be configured to be minimally invasively inserted as described herein, thus increasing the number of recording arrays that may be feasibly applied to a subject. In some embodiments, the one or more recording arrays 505 may comprise a plurality of recording arrays 505. In some embodiments, multiple recording arrays 505 may be placed at the same region of the brain. In some embodiments, the recording arrays 505 may be placed at the different regions of the brain. For example, recording arrays 505 may be placed in two or more adjacent regions of the brain. In another example, recording arrays 505 may be placed in two or more non-adjacent regions of the brain. In some embodiments, the recording arrays 505 may be re-positionable by minimally invasive techniques after initial placement in order to improve the data being collected. In some embodiments, the recording arrays 505 may be removable from the subject by minimally invasive techniques after a period of use. Accordingly, the system 500 may be able to collect a greater volume of electrophysiological data and/or a more diverse set of electrophysiological data (i.e., representing a great number of regions of the brain) over conventional systems.

The at least one stimulation array 510 may comprise one or more stimulation electrodes capable of stimulating nervous tissue with electrical signals delivered to a target stimulation site. The stimulation array 510 may be configured to be surgically inserted to the target stimulation site of the nervous system by conventional techniques. In some embodiments, the target stimulation site may be at or within the spinal cord. In some embodiments, the target stimulation site may be at or within the spinal nerve roots. In some embodiments, the stimulation array 510 comprises a high-density array that enables greater spatial resolution as described herein with respect to the recording array 505. For example, the stimulation array 510 may comprise any structure, features, and arrangement as described herein with respect to the recording array 505 while also being configured to stimulate a target tissue. In some embodiments, the at least one stimulation array 510 comprises a single stimulation array 510. In some embodiments, the at least one stimulation array 510 comprises a plurality of stimulation arrays 510. In some embodiments, multiple stimulation arrays 510 may be placed at the same region of the nervous tissue and/or at different regions of the nervous tissue.

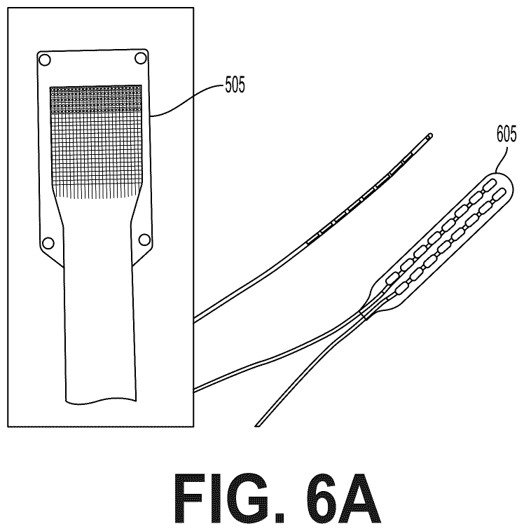

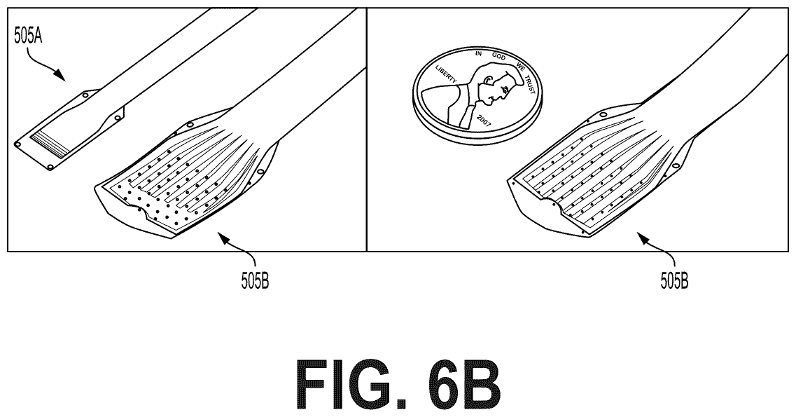

Referring now to FIG. 6A, a comparison of the stimulation array 510 of FIG. 5 to a conventional stimulation electrode array is depicted in accordance with an embodiment. Further, FIG. 6B illustrates a comparison between a 529-channel electrode array 505A and a 1,024-channel electrode array 505B in accordance with the embodiments described herein. The recording array 505A as shown can comprise about 500 electrodes arranged in a density of about 100 electrodes/cm′ or more. In one illustrative embodiment, the recording array 505A can include 529 electrodes. The recording array 505B as shown can comprise about 1,000 electrodes arranged in a density of about 100 electrodes/cm² or more. In contrast, a conventional stimulation electrode array 605 may comprise 16 electrodes of a diameter of 1 mm or greater spaced linearly by several millimeters. Accordingly, the stimulation array 505 may be able to stimulate the nervous tissue to alter its electrophysiological activity with greater accuracy and spatial resolution than a conventional stimulation electrode array 605. Still further, the stimulation array 505 may be able to alter the electrophysiological activity of the nervous tissue and the state thereof in manners not possible by conventional stimulation arrays for SCS. Nonetheless, it can be understood that in some embodiments, the stimulation array 510 of the system 500 may comprise a conventional lead having features and/or functions as would be understood by a person having an ordinary level of skill in the art.

The control unit 515 may be in electrical communication with the one or more recording arrays 505 and the at least one stimulation array 510 in order to perform closed-loop SCS. In some embodiments, the control unit 515 includes a processor and a memory such as a non-transitory, computer-readable medium storing instructions for performing closed-loop SCS. It can be understood that the control unit 515 may comprise any number of components of the neural device 110 as described herein with respect to FIGS. 1-3 (e.g., the electrode-amplifier stage 112, an analog front-end stage 114, an ADC stage 116, a DSP stage 118, and/or a transceiver stage 120) and may comprise any of the features and/or functions as described with respect to the neural device 110.

Referring once again to FIG. 5, in some embodiments, the system 500 may further comprise one or more inertial sensors 520 in electrical communication with the control unit 515. The one or more inertial sensors 520 may be applied to the subject in a variety of manners. In some embodiments, the inertial sensors 520 are housed with the control unit 515. In some embodiments, the inertial sensors 520 are housed with the recording array 505 and/or the stimulation array 510. In some embodiments, the inertial sensors 520 are applied to the subject as a separate component. In some embodiments, the inertial sensors 520 are implanted within the body of the subject. In some embodiments, the inertial sensors 520 are affixed externally on the body of the subject. In some embodiments, the inertial sensors 520 are encased in a housing or material to insulate and/or stabilize the inertial sensors 520 are the site of placement. In some embodiments, the inertial sensors are located at or near the nervous tissue in order to accurately provide orientation information related to the spinal cord. It can be noted that it may not be required that the inertial sensors 520 be in contact with the nervous tissue.

The inertial sensors 520 are configured to detect an orientation and/or a movement state of the subject in real time. In some embodiments, the inertial sensors 520 sense inertial information that may be processed by the control unit 515 and/or by the inertial sensors 520 to determine an orientation of the subject and/or a movement state of the subject. For example, the inertial sensors 520 may determine an orientation of the subject from a list consisting of standing, sitting, supine, prone, kneeling, leaning, or additional orientations generally associated with day-to-day life or specific activities. In some embodiments, the inertial sensors 520 may determine a movement state of the subject from a list consisting of still, walking, jogging, running, or additional movement states generally associated with day-to-day life or specific activities.

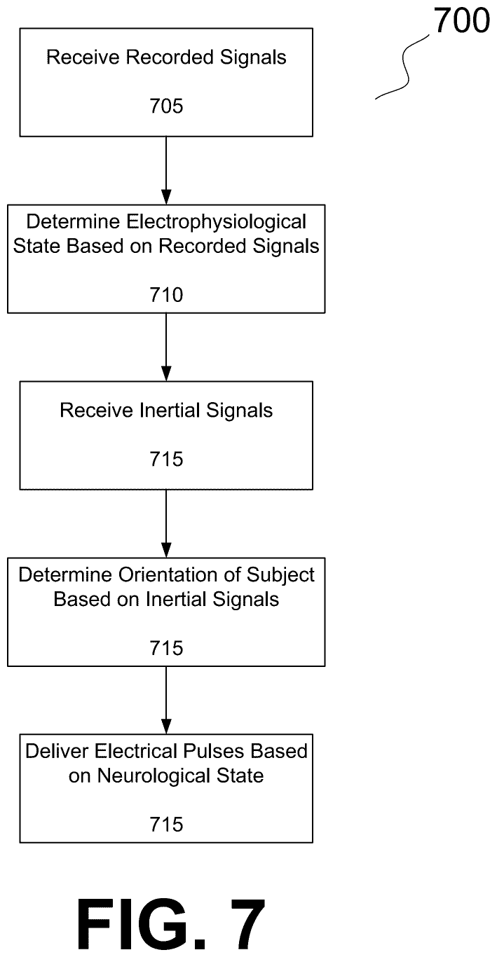

Turning now to FIG. 7, a flow diagram of an illustrative computer-implemented method 700 for performing SCS by the system 500 is depicted in accordance with an embodiment. For example, the method 700 may be carried out by the processor of the control unit 515 upon execution of the instructions stored on the memory. The method 700 comprises receiving 705 one or more recorded signals (i.e., collected at the target recording site) from the recording array(s) 505, determining 710 an electrophysiological state of the brain based on at least the one or more recorded signals, receiving 715 one or more inertial signals from the one or more inertial sensors, determining 720 an orientation of the subject based on the one or more inertial signals, and selectively delivering 725 electrical stimulation to the target stimulation site via the stimulation array(s) 510 based on the determined neurological state and/or the determined orientation of the subject.

As shown in FIG. 3, each recording array 505 may electrically communicate with the control unit 515 to communicate the recorded signals thereto. It can be understood that the control unit 515 may continuously receive 705 recorded signals from the recording array 505 at a temporal resolution suitable for determining a neurological state of the brain as would be known to a person having ordinary skill in the art. Accordingly, the control unit 515 may determine 710 and update the determination of the neurological state of the brain over time.

The control unit 515 may determine 710 the neurological state of the brain in a variety of manners as would be known to a person having an ordinary level of skill in the art. In some embodiments, the control unit 515 may recognize features and/or patterns in the recorded signals to determine the electrophysiological state. For example, the control unit 515 may access a database comprising information related to signal features and/or patterns associated with various electrophysiological states and/or symptoms thereof. In some embodiments, the database may be stored on the memory or another location accessible to the control unit 515. The control until 515 may compare the recorded signals to the information in the database in order to identify the electrophysiological state.

As shown in FIG. 3, the control unit 515 may electrically communicate with each stimulation array 510 to operate the electrodes to deliver 715 the electrical stimulation. It can be understood that the electrical stimulation may comprise one electrical pulse or a plurality of electrical pulses having varying parameters. Furthermore, the electrical stimulation may be delivered 715 at a temporal resolution suitable for affecting an electrophysiological state of the nervous tissue and/or symptoms thereof as would be known to a person having ordinary skill in the art. In some embodiments, the control until 515 may access a database comprising a set of pulse parameters directed to treat a specific electrophysiological state and/or symptoms thereof. In some embodiments, the database may be stored on the memory or another location accessible to the control unit 515.

In some embodiments, the electrical stimulation may be configured to electrically stimulate the nervous tissue to change the electrophysiological state thereof. For example, the electrical stimulation may result in reduction or elimination of one or more symptoms associated with the electrophysiological state. In some embodiments, the set of electrical stimulation may be configured to electrically stimulate the brain to maintain an electrophysiological state of the brain. For example, the electrical stimulation may prevent onset of one or more symptoms associated with a specific condition and/or reduce occurrence of the one or more symptoms.

In some embodiments, the control unit 515 may be configured to determine 710 or detect a single electrophysiological state. In some embodiments, the control unit 515 may be configured to determine 710 the electrophysiological state from a plurality of possible electrophysiological states.

In some embodiments, an electrophysiological state may be a condition or symptom associated with a neurological disease or disorder and/or injuries associated with the spinal cord. As such, the control unit 515 may determine 710 whether the condition or symptom is actively presenting in the subject. For example, the neurological state may be one or more of chronic pain, autonomic dysfunction, autonomic dysfunction with dominant components of cardiac arrhythmia or intermittent hypotension, postural instability, gait dysfunction, multiple system atrophy, shaking, tremors, paralysis, blood pressure control, heart rate control, and postural control.

In some embodiments, an electrophysiological state may be a current disposition of a neurological disorder or condition. In other words, for conditions that may have varying states (e.g., chronic pain and tremors), the control unit 515 may determine 710 the current disposition of the subject, i.e., whether the subject is experiencing symptoms of the condition and/or onset of symptoms. In the case of chronic pain, the neurological state may comprise a degree or level of pain. Furthermore, the neurological state may be a level of pain registered by the brain but at an intensity not yet exceeding the threshold of conscious awareness, i.e., imminent onset of pain. Accordingly, the control unit may identify there is a window to change stimulation parameters to prevent conscious sensation of pain. For example, in the case of paralysis, the electrophysiological state may comprise time varying signals reflecting intentions, such as intentions to move a limb purposefully, to execute motions for walking, or coordinate postural control. Identifying such signals would be useful in a variety of different applications, including neural decoding for motor neuroprosthetic function.

In some embodiments, an electrophysiological state may be a status of the subject that correlates with the conditions or symptoms of a neurological condition. For example, where particular conditions or symptoms of the neurological condition affect movement and/or postural control, the control unit 515 may determine 710 whether the subject is intending to move limbs, thus indicating walking, jogging, running, or changing posture.

It can be understood that while various exemplary conditions, symptoms, diseases, and disorders are described herein, the method 700 may be applied to various additional conditions, symptoms, diseases, and disorders as would be apparent to a person having an ordinary level of skill in the art.

It can also be understood that the target recording site(s) and the target stimulation site(s) may be selected based on a target symptom, condition, and/or disorder. In some embodiments, the target condition is selected from the group consisting of chronic pain, autonomic dysfunction, multiple system atrophy, shaking, tremors, and paralysis.

In some embodiments, the target condition is tremors, shaking, or paralysis and the target stimulation site is the motor cortex. In some embodiments, the target disorder is chronic pain, and the target stimulation site is one of a nervous tissue detecting pain, or a nervous system tissue conducting pain signals to the brain, or a nervous system tissue processing pain signals.

The devices, systems, and methods as described herein are not intended to be limited in terms of the particular embodiments described, which are intended only as illustrations of various features. Many modifications and variations to the devices, systems, and methods can be made without departing from their spirit and scope, as will be apparent to those skilled in the art.

In some embodiments, the system 500 may be configured to communicate with an external device. For example, the control unit 515 may communicate with an external device in a similar manner as the neural device 110 communicates with the external device 130 as described with respect to FIG. 1.

In some embodiments, the control unit 515 may communicate with external devices to program the control unit 515, re-program the control unit 515, and/or to receive software updates.

In some embodiments, the control unit 515 may communicate with external devices to receive information for the database and/or to expand or update the information in the database, e.g., to improve sensing and/or expand sensing to additional symptoms or conditions. For example, the control unit 515 may receive additional patterns or features for recognition in the recorded signals and/or additional pulse patterns to deliver to the deep brain to treat a symptom or disorder. It can be understood that a particular system 500 may be tailored to a particular disorder and/or a set of symptoms associated with the subject, and thus may be programmed and/or re-programmed as such.

### Data Processing Systems for Implementing Embodiments Herein

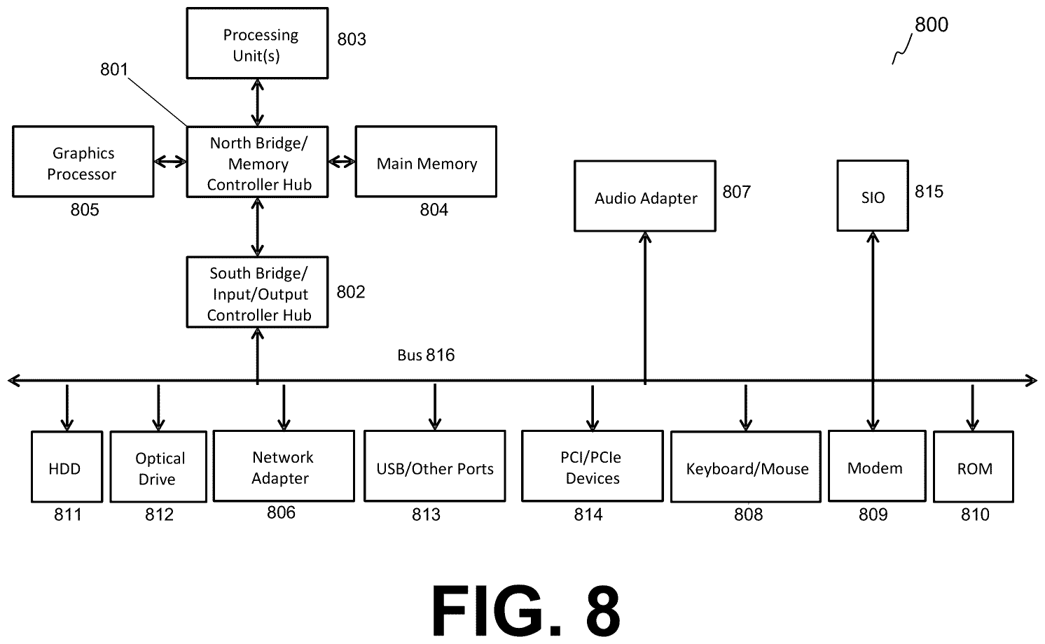

FIG. 8 illustrates a block diagram of an exemplary data processing system 800 in which embodiments are implemented. The data processing system 800 is an example of a computer, such as a server or client, in which computer usable code or instructions implementing the process for illustrative embodiments of the present invention are located. In some embodiments, the data processing system 800 may be a server computing device. For example, data processing system 800 can be implemented in a server or another similar computing device operably connected to a neural device 110 or a system 500 as described above. The data processing system 800 can be configured to, for example, transmit and receive information related to a patient and/or a treatment plan with the system 500.

In the depicted example, data processing system 800 can employ a hub architecture including a north bridge and memory controller hub (NB/MCH) 801 and south bridge and input/output (I/O) controller hub (SB/ICH) 802. Processing unit 803, main memory 804, and graphics processor 805 can be connected to the NB/MCH 801. Graphics processor 805 can be connected to the NB/MCH 801 through, for example, an accelerated graphics port (AGP).

In the depicted example, a network adapter 806 connects to the SB/ICH 802. An audio adapter 807, keyboard and mouse adapter 808, modem 809, read only memory (ROM) 810, hard disk drive (HDD) 811, optical drive (e.g., CD or DVD) 812, universal serial bus (USB) ports and other communication ports 813, and PCI/PCIe devices 814 may connect to the SB/ICH 802 through bus system 816. PCI/PCIe devices 814 may include Ethernet adapters, add-in cards, and PC cards for notebook computers. ROM 810 may be, for example, a flash basic input/output system (BIOS). The HDD 811 and optical drive 812 can use an integrated drive electronics (IDE) or serial advanced technology attachment (SATA) interface. A super I/O (SIO) device 815 can be connected to the SB/ICH 802.

An operating system can run on the processing unit 803. The operating system can coordinate and provide control of various components within the data processing system 800. As a client, the operating system can be a commercially available operating system. An object-oriented programming system, such as the Java programming system, may run in conjunction with the operating system and provide calls to the operating system from the object-oriented programs or applications executing on the data processing system 800. As a server, the data processing system 800 can be an IBM® eServer™ System® running the Advanced Interactive Executive operating system or the Linux operating system. The data processing system 800 can be a symmetric multiprocessor (SMP) system that can include a plurality of processors in the processing unit 803. Alternatively, a single processor system may be employed.

Instructions for the operating system, the object-oriented programming system, and applications or programs are located on storage devices, such as the HDD 811, and are loaded into the main memory 804 for execution by the processing unit 803. The processes for embodiments described herein can be performed by the processing unit 803 using computer usable program code, which can be located in a memory such as, for example, main memory 804, ROM 810, or in one or more peripheral devices.

A bus system 816 can be comprised of one or more busses. The bus system 816 can be implemented using any type of communication fabric or architecture that can provide for a transfer of data between different components or devices attached to the fabric or architecture. A communication unit such as the modem 809 or the network adapter 806 can include one or more devices that can be used to transmit and receive data.

Those of ordinary skill in the art will appreciate that the hardware depicted in FIG. 8 may vary depending on the implementation. Other internal hardware or peripheral devices, such as flash memory, equivalent non-volatile memory, or optical disk drives may be used in addition to or in place of the hardware depicted. Moreover, the data processing system 800 can take the form of any of a number of different data processing systems, including but not limited to, client computing devices, server computing devices, tablet computers, laptop computers, telephone or other communication devices, personal digital assistants, and the like. Essentially, data processing system 800 can be any known or later developed data processing system without architectural limitation.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “patient” and “subject” are interchangeable and refer to any living organism which contains neural tissue. As such, the terms “patient” and “subject” may include, but are not limited to, any non-human mammal, primate or human. A subject can be a mammal such as a primate, for example, a human. The term “subject” includes domesticated animals (e.g., cats, dogs, etc.); livestock (e.g., cattle, horses, swine, sheep, goats, etc.), and laboratory animals (e.g., mice, rabbits, rats, gerbils, guinea pigs, possums, etc.). A patient or subject may be an adult, child or infant.

The term “tissue” refers to any aggregation of similarly specialized cells which are united in the performance of a particular function.

The term “disorder” is used in this disclosure to mean, and is used interchangeably with, the terms “disease,” “condition,” or “illness,” unless otherwise indicated.

The term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

1. A system for performing spinal cord stimulation on a patient, the system comprising:
one or more recording arrays configured to be minimally invasively inserted to a target recording site of a brain of the patient, each recording array comprising a plurality of recording electrodes having a spacing of less than about 1 mm therebetween, each recording electrode having a diameter of less than about 1 mm and configured to record electrical signals from the target recording site;
at least one stimulation array configured to be inserted to a target stimulation site proximate to nervous tissue of the patient, each stimulation array comprising a plurality of stimulation electrodes, each stimulation electrode configured to deliver electrical stimulation to the target stimulation site;
one or more inertial sensors configured to detect at least one of an orientation and a movement state of the patient;
a processor; and
a non-transitory, computer-readable medium storing instructions that, when executed, cause the processor to:
receive, via the one or more recording arrays, one or more recorded signals from the target recording site,
determine an electrophysiological state of the brain based on at least the one or more recorded signals,
receive one or more inertial signals from the one or more inertial sensors,
determine the at least one of an orientation and a movement state of the patient based on the one or more inertial signals, and
selectively deliver, via the at least one stimulation array, electrical stimulation to the target stimulation site based on one or more of the determined electrophysiological state and the determined at least one of an orientation and a movement state of the patient, wherein the electrical stimulation is configured to electrically stimulate the nervous tissue.

2. The system of claim 1, wherein each of the one or more recording arrays is a thin-film electrode array.

3. The system of claim 1, wherein each of the one or more recording arrays and the at least one stimulating array is a two-dimensional electrode array.

4. The system of claim 1, wherein each of the one or more recording arrays has a spatial density of at least about 100 electrodes/cm².

5. The system of claim 1, wherein each of the one or more recording arrays comprises at least about 100 electrodes.

6. The system of claim 5, wherein each of the one or more recording arrays comprises about 1,024 electrodes.

7. The system of claim 1, wherein the electrophysiological state is selected from chronic pain, autonomic dysfunction, autonomic dysfunction with dominant components of cardiac arrhythmia or intermittent hypotension, postural instability, gait dysfunction, multiple system atrophy, shaking, tremors, and paralysis.

8. The system of claim 1, wherein the electrophysiological state is tremors, shaking, or paralysis, and the target stimulation site is the spinal cord.

9. The system of claim 1, wherein the electrophysiological state is pain, and the target stimulation site is a nervous tissue detecting pain.

10. The system of claim 9, wherein the electrophysiological state is pain at a level below a threshold of conscious awareness for the patient.

11. The system of claim 1, wherein the electrophysiological state is intention to move, unconscious postural control, or autonomic dysfunction and the target stimulation site is the spinal cord to trigger skeletal motor activity, postural control, or heart rate stability.

12. A computer-implemented method for performing spinal cord stimulation on a patient, the method comprising:
receiving one or more recorded signals from a target recording site via one or more recording arrays positioned at the target recording site, each recording array comprising a plurality of recording electrodes having a spacing of less than about 1 mm therebetween, each recording electrode having a diameter of less than about 1 mm;
receiving one or more inertial signals indicative of at least one of an orientation and a movement state of the patient via one or more inertial sensors;
determining an electrophysiological state of a brain of the patient based on at least the one or more recorded signals;
determining the at least one of an orientation and a movement state of the patient based on the one or more inertial signals; and
selectively delivering, based on one or more of the determined electrophysiological state and the determined at least one of an orientation and a movement state, electrical stimulation to a target stimulation site via at least one stimulation array positioned at the target stimulation site to electrically stimulate the target stimulation site, wherein the target stimulation site is located at one of a spinal cord and spinal nerve roots of the patient.

13. The method of claim 12, wherein each of the one or more recording arrays is a thin-film electrode array.

14. The method of claim 12, wherein each of the one or more recording arrays and the at least one stimulating array is a two-dimensional electrode array.

15. The method of claim 12, wherein each of the one or more recording arrays has a spatial density of at least about 100 electrodes/cm².

16. The method of claim 12, wherein each of the one or more recording arrays comprises at least about 100 electrodes.

17. The method of claim 16, wherein each of the one or more recording arrays comprises about 1,024 electrodes.

18. The method of claim 12, wherein the electrophysiological state is selected from chronic pain, autonomic dysfunction, autonomic dysfunction with dominant components of cardiac arrhythmia or intermittent hypotension, postural instability, gait dysfunction, multiple system atrophy, shaking, tremors, and paralysis.

19. The method of claim 12, wherein the electrophysiological state is tremors, shaking, or paralysis and the target stimulation site is the spinal cord.

20. The method of claim 12, wherein the electrophysiological state is pain, and the target stimulation site is a nervous tissue detecting pain.

21. The method of claim 20, wherein the electrophysiological state is pain at a level below a threshold of conscious awareness for the patient.

22. The method of claim 12, wherein the electrophysiological state is intention to move, unconscious postural control, or autonomic dysfunction and the target stimulation site is the spinal cord to trigger skeletal motor activity, postural control, or heart rate stability.
